(19) United States
(12) Patent Application Publication
(10) Pub. No.: US 2006/0081245 A1
(43) Pub. Date: Apr. 20, 2006

(54) Surgical tube guard

(76) Inventor: Kion H. Gould, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/240,808

(22) Filed: Sep. 30, 2005

Related U.S. Application Data
(60) Provisional application No. 60/615,219, filed on Oct. 1, 2004.

(51) Int. Cl. A61M 16/00; A62B 9/06

(52) U.S. Cl. 128/200.26; 128/207.14; 128/207.15; 128/848

(57) ABSTRACT

A bite-block device for preventing biting of air tubes, such as laryngeal mask airway (LMA) tubes by human teeth is disclosed. The device is composed of a generally cylindrical body, fabricated from a biologically compatible polymer. A hole is present through the long axis of the body. The device also extends in a plane perpendicular to the long axis of the body. Attached to this plane is a coupling device intended for securing an LMA tube to the bite-block. The bite-block is positioned within a patient's mouth against the cheek, with the plane perpendicular to the long axis pressed against the lip. Deployed in this fashion, the bite block engages the molars, bicuspids, and cuspids, preventing contact of the teeth with an LMA tube.

### CROSS-REFERENCE TO RELATED APPLICATIONS

This application claims the benefit of U.S. provisional application No. 60/615,219 by Kion H. Gould entitled “Surgical Tube Guard,” filed on Oct. 1, 2004.

### BACKGROUND OF THE INVENTION

1. Field of the Invention

The present invention relates to a surgical apparatus and, in particular, concerns a guard for protecting tubes which are inserted into a patient's mouth during surgery.

2. Description of the Related Art

The laryngeal mask airway (LMA) is a medical device that has found widespread utility in anesthetic practice and emergency medicine. The device is used by surgeons and rescue personnel to ensure that a patient is able to breathe during medical procedures. The device consists of a wide tube with a connector at one end and an elliptical, inflatable mask at the opposite end. The mask is passed through the passage which extends from the mouth and nasal cavity (the pharynx) until resistance is felt. At this point, the mask is positioned just above the vocal cords at the upper part of the passage connecting the pharynx and the windpipe (the larynx). When inflated, the mask forms a low pressure seal around the opening of the larynx. The patient is then ventilated using a mechanical ventilator attached to the end of the LMA tube emerging from the mouth.

During the use of the LMA, however, patients often bite down on the LMA tube. This action is undesirable for several reasons. The first reason is the obvious danger to the patient due to blockage of the breathing airway passage. The second is that biting the LMA tube can result in damage to the tube which can puncture or sever the tube, thereby compromising the ability of the tube to deliver oxygen to the patient.

In ventilation applications, bite-blocks are sometimes used to address these problems. There are two general bite block designs commonly employed. In the first configuration, the bite block is designed to engage the incisors over a limited area. One implementation of this configuration utilizes an elongated, U-shaped channel, corresponding to the size and shape of an air tube. The channel is of slightly larger dimensions than the tube, allowing the tube to fit within the channel. The tube is placed within the channel and surrounded on three sides. The parallel sides of the unshaped channel are longer than the diameter of the tube so that in the process of biting, the incisors contact the channel rather than the tube. The bite-block is located within the center of the LMA device, which in turn is centered in the mouth. Examples of this type of tube are shown in U.S. Pat. Nos. 6,474,332 and 5,355,874.

This design presents a number of practical problems during use. By using the walls of the channel containing the air tube as a bite block, the only a small area of material is in contact with the incisors. This will result in high forces exerted over a small area, giving rise to large pressures on the bite block when the patient bites down on it, which can lead to failure of the block. Further, a three-sided bite block structure is mechanically weaker than a structure which fully surrounds the air tube. For these reasons, extra material or stronger plastics are required to raise the bite-block strength, increasing the cost of the device.

Moreover, the large size of the air tube bite-block and its position obstruct the oral cavity. This obstruction complicates the use of other devices, such as suction devices which are commonly inserted into the mouth during oral procedures. Additionally, positioning the bite block in the center of the mouth makes it more difficult for the physician to see into the oral cavity.

Yet a further difficulty is that these bite blocks use elongated U-shaped channels to hold the LMA tube over relatively long lengths. As such, this requires the tube to be inserted and secured within the bite block along a relatively long length after the bite block has been installed in the patient's mouth. Consequently, this installation can be difficult and time consuming for the treating medical personnel. Similarly, removal of the air tube from the bite block can also be more complicated.

Other configurations of bite blocks for use with air tubes have also been developed. Some incorporate flanges that engage with the patients teeth (See, e.g., U.S. Pat. No. 6,755,191) and other designs comprise simple bite-blocks that prevent the jaws from closing but are not otherwise coupled to the air tube (See, e.g., U.S. Pat. No. 5,655,519). These designs are still complicated to use and often result in greater obstruction in the patient's mouth.

From the foregoing, it is apparent that there is a need for an improved bite-block and air tube holder. In particular, there is a need for a bite-block device to be used in LMA applications that protects the air tube, is simple to use and provides less obstruction in the patient's mouth.

### SUMMARY OF THE INVENTION

The aforementioned needs are satisfied by the LMA bite-block apparatus of the present invention which, in one aspect, comprises a bite structure that is dimensioned so as to be interposed between the patient's molars on one side of the mouth to inhibit the patient from closing their mouth. In this aspect, the apparatus further includes a capture opening that is sized so as to receive an LMA air tube. In one particular implementation, the capture opening is open on one side so as to facilitate installation and removal of the LMA air tube from the capture opening.

In one implementation, the bite-block apparatus further includes a flange that is adapted to be positioned in front of the patient's lips so as to allow the bite block to be secured in position. In this implementation, the capture opening is positioned adjacent the flange and extends outwardly such that the capture opening is generally removed from the patient's mouth which facilitates installation and removal of the tube from the bite-block apparatus.

In one specific implementation, the bite-block structure is tapered from a first end to a second end such that the bite-block structure, when positioned between the patient's molars prevents the patient from closing their molars. At the first end, the flange is positioned so as to extend generally perpendicular to the direction of the bite block structure and the capture opening is formed adjacent one side of the flange. This results in a compact bite-block apparatus which reduces the obstruction within the patient's mouth. In one embodiment, the bite-block structure has a through going hole that can receive a supplemental suction device.

Hence, the apparatus described herein provide for an easier to use, less obstructing device that protects air tubes, such as those used in LMA application from being damaged by the patient's teeth. These and other objects and advantages will become more apparent from the following description taken in conjunction with the accompanying drawings.

### BRIEF DESCRIPTION OF THE DRAWINGS

Other features and advantages of the invention will be apparent from the following description taken in connection with the accompanying drawings, wherein:

FIG. 1 is a perspective view of one of the preferred embodiments of the bite-block apparatus;

FIG. 2 is a front side view of a bite-block apparatus of FIG. 1;

FIG. 3 is a top view of the bite-block apparatus of FIG. 1;

FIG. 4 illustrates the bite-block apparatus of FIG. 1 and an LMA airway tube into which the bite-block is to be attached;

FIG. 5A and 5B illustrates the bite-block apparatus of FIG. 1 and an LMA airway tube into which the bite-block is attached by a coupling mechanism;

FIG. 6 illustrates a perspective view of the bite-block apparatus connected to the LMA airway tube of FIG. 1 inserted into place within the patient's mouth;

FIG. 7 illustrates a front view of the bite-block apparatus connected to the LMA airway tube of FIG. 6 inserted into place within the patient's mouth, demonstrating line of sight into the mouth.

### DETAILED DESCRIPTION OF THE ILLUSTRATED EMBODIMENT

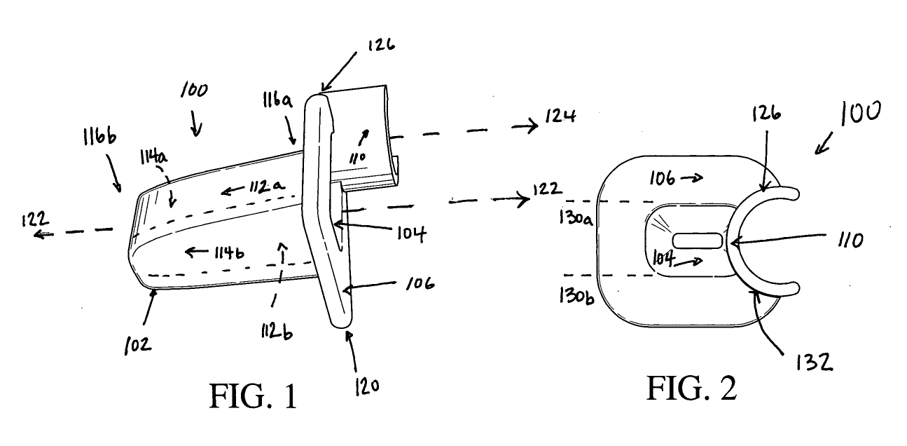

FIG. 1 illustrates one embodiment of a perspective view of an LMA bite-block device 100. Generally, the device 100 is designed for partial placement within a patient's mouth so as to protect an inserted air tube, such as an LMA tube, from damage or occlusion resulting from the patient biting the tube. The device includes a bite-block body 102, a central cavity 104, a flange 106, and a tube coupling mechanism 110.

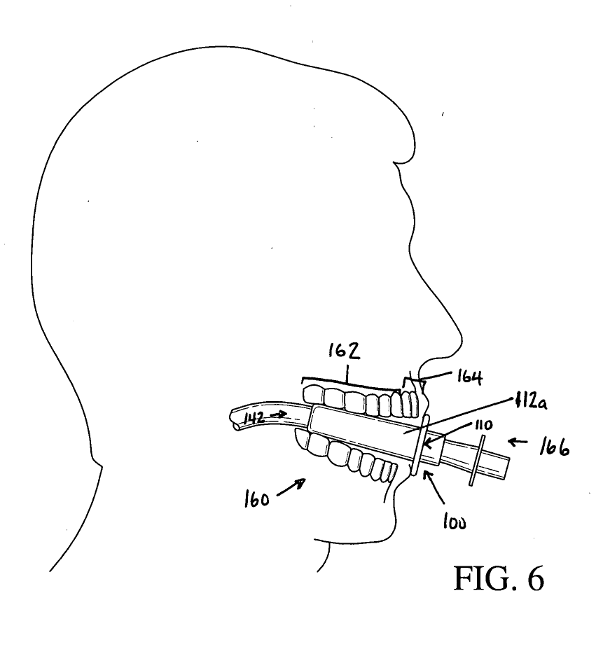

The bite-block 100 possesses a generally elongate bite-block body 102, comprising top and bottom walls 112a and 112b, side walls 114a and 114b, and a first end 116a and a second end 116b. The bite-block body 102 is preferably dimensioned such that, when inserted between the patient's molars, the patient's front teeth are separated by a distance that is greater than the width of the air tube. In the embodiment illustrated in FIG. 1, the top and bottom walls 112a and 112b are approximately ¾″ wide by 1¼″ long. The side walls extend a width, on average, of approximately 9/16″ along a similar length but are tapered, as will be described below. The top and bottom walls 112a and 112b of the bite-block body 102 are substantially flat, configured to engage with the teeth in a manner to be described in more detail below in respect to FIG. 6.

The bite-block body 102 is tapered from the first end 116a to the second end 116b at an angle between 3 and 4 degrees. As the oral cavity becomes wider towards the lips when the mouth is opened and the bite-block device is inserted into the mouth at the second end 116b, the tapered shape of the bite-block body 102 promotes accommodation of the device 100 within the mouth and also matches the general position of the patient's teeth that are engaging the bite-block body 102.

It will be appreciated that the bite-block body 102 design illustrated is simply one embodiment and that a number of variations of this design may be made by those skilled in the art without departing from the scope of the present teachings. In one aspect, the second end 116b of the bite-block body 102 may be rounded in order to substantially reduce any sharp ends which may cause discomfort for the patient. In another aspect, the cross-section of the bite-block body 102 may be constructed in a non-rectangular geometry without departing from the scope of the present teaching.

As illustrated in FIG. 1, the bite-block body 102 may contain a bite-block cavity 104 formed therein. In particular, FIGS. 1 and 2 show the cavity 104 defined by bite and side walls 112a, 112b, 114a, and 114b of the bite-block body 102, generally centered in the cross-section of the bite-block body 102. The cavity 104 is tapered from the first end 116a to the second end 116b and continuous through the length of the bite-block device 100. The cavity 104 functions as a breathing airway, should the bite-block device 100 become lodged in a patient's throat during use, enhancing the safety of the device 100. Further, the cavity 104 also allows the use of supplemental suction via the cavity 104 and also facilitates visual access to the interior of the patient's mouth.

A flange 106 extends outward from the first end 116a of the bite-block body 102. In the embodiment illustrated in FIG. 1, the flange is generally rectangular in shape and may extend approximately ⅜″ beyond the bite-block body 102. In another aspect, the corners 120 of the flange 106 may be rounded to make the bite-block 100 more comfortable and les prone to abrading or injuring the patient. In this embodiment, the flange 106 is oriented substantially perpendicular to the axis 122 of the bite-block body 102. The cavity 104 passes through the flange 106, leaving an unobstructed path through the bite-block 100 from the first end 116a to the second end 116b. The flange 106 is configured so as to engage the patient's lips so as to inhibit entry of the bite-block 100 into a patient's mouth, in a manner that will be described below in more detail with reference to FIG. 6. Further, the flange 106 provides an exterior surface to which tape can be used to secure the bite block 100 in the desired position interposed between the patient's teeth.

FIG. 1 also illustrates a coupling mechanism 110 for the bite-block device 100. The coupling mechanism 110 in this implementation comprises a substantially semi-cylindrical shape defining a capture opening whose axis 124 is generally parallel with the axis 122 of the bite-block body 102. A base 126 of the coupling mechanism 110 is attached to the flange 106, adjacent to one of the side walls 114a and 114b of the bite-block body 102. The coupling mechanism 110 secures the bite-block device 100 to an air tube of an LMA device in a manner to be described below in greater detail with respect to FIG. 4.

FIG. 2 illustrates a view of the bite-block device 100 facing the first end 116a of the bite-block 100. In the embodiment illustrated, the top and bottom walls 112a, 112b respectively define planes 130a, 130b. The patient's teeth (not shown), engage the bite-block body 102 substantially perpendicular to the horizontal planes 130a and 130b at a location behind the flange 106. Positioning the bite-block device 100 in this fashion orients the coupling mechanism 110 to one side of the bite-block body 102 in front of the teeth. Additionally shown in FIG. 2, the coupling mechanism 110 is connected to the flange 106 over a distance of approximately ¾ of the radius of the mechanism 110. This provides for substantially secure attachment of the coupling mechanism 110 to the flange 106. FIG. 2 further illustrates that the coupling mechanism 110 is positioned adjacent to, but not substantially obstructing, the cavity 104. The outer surface 132 of the coupling mechanism 110 may also serve as an attachment point for adhesive tapes which may be used to further secure LMA tubes inserted into the coupling mechanism 110. Alternatively, the coupling mechanism 110 alone may be sufficiently biased or sized so as to return the LMA tube therein without the use of the tape.

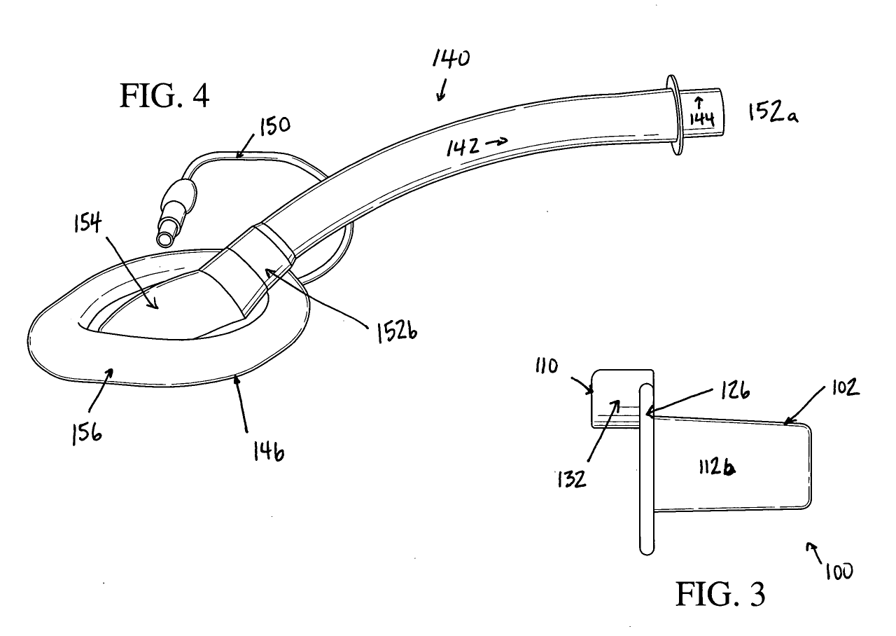

FIG. 3 shows a side view of the bite-block device 100. As in one illustrated embodiment, the coupling mechanism 110 may extend approximately ⅜″ outward from the flange 106. This length provides an area with which to secure the device 100 to an LMA device. As the coupling mechanism 110 extends outward from the flange 105, the medical personnel can place the tube in the coupling mechanism 110 exterior to the patient's mouth which greatly facilitates the use of the bite block device 100.

FIG. 4 illustrates an exemplary LMA device 140. The device 140 consists of an LMA tube 142, a connection device 144, an inflatable mask 146, and a mask inflation device 150. The LMA tube 142 possesses a generally circular cross-section between a first end 152a and a second end 152b. A connector 144 is joined to the LMA tube 142 at the first end 150. The connector 144 is designed to engage with a mechanical ventilation machine in a generally known manner so as to create a substantially airtight seal and allow gas flowing from the ventilator to enter the LMA tube 142. The inflatable mask 146 comprises an elliptical mask core 150 attached to and encircled by an inflatable ring 156. The LMA tube 142 is connected to the mask core 150 at a second end 152b of the tube 142.

In use, the LMA device 142 is passed into the patient's mouth, second end 152b first, through the passage which extends from the mouth and nasal cavity until the mask is positioned just above the vocal cords at the upper part of the passage connecting the pharynx and the windpipe (the larynx). When inflated with the mask inflation device 150, the ring 156 forms a low pressure seal around the opening of the windpipe. This seal substantially allows only the gas within the LMA tube 142 to pass into the patient's breathing airways.

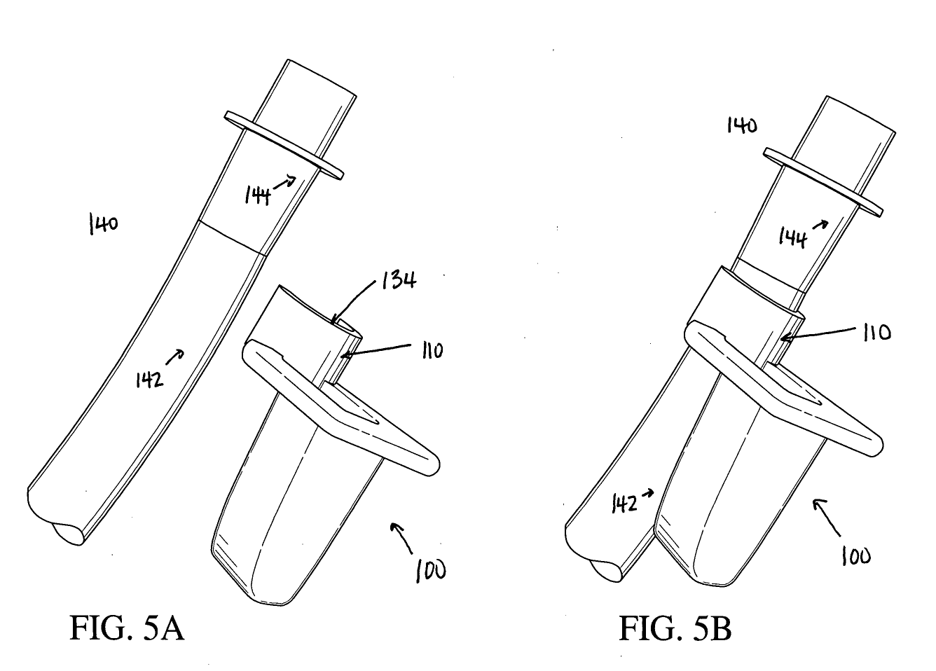

FIGS. 5A-5B illustrate the LMA tube 142, connector 144, and bite-block 100, and the process of attaching the LMA device 140 to the bite-block 100. FIG. 5A illustrates that the bite-block 100 is placed adjacent to the LMA tube 142 near the connector 144 with the coupling mechanism 110 facing the tube 142. The coupling mechanism 110 is selected to be of predetermined radius so as to be substantially equal to that of the LMA tube 142. The coupling mechanism 110 is pressed against the LMA tube 142 with sufficient force so as to elastically deform the mechanism 110 sufficiently so as to accommodate the tube 142 within the inner circumference 134 of the coupling mechanism 110. As a result of this process, in the coupled position illustrated in FIG. 5B, the coupling mechanism 110 exerts a compressive or functional force on the LMA tube 142 which acts to substantially constrain the position of the tube 142 with respect to the bite-block 100 and enhance the safe operation of the device 100. The process described above is reversed in order to remove the tube 142 from the bite-block 100. Adhesive may optionally be used to further secure the tube in the coupling mechanism 106.

FIG. 6 illustrates a side view of an LMA device 140 which has been inserted into a patient's oral cavity 160, wherein the bite-block 100 has been attached to the LMA tube 142. As shown in FIG. 6, the top and bottom walls 112a and 112b of the bite-block 100 engage a plurality of the patient's back teeth 162. This rearward engagement forces the patient's front teeth 164 apart at the opening of the mouth 166 so that the front teeth 164 are inhibited from contacting or biting the LMA tube 142. This protects the tube from blockage by biting, enhancing the safety of using the LMA device 140, as well as extending the lifetime of the LMA tube 142. Additionally, engagement of the teeth 162 by the bite-block 100 over a broad area results in low biting forces upon the bite-block 100, reducing the likelihood of bite-block damage or failure. Further, the bite-block 100 is coupled to the LMA tube 142 outside the oral cavity 160, enhancing ease of attachment and removal of the bite-block 100. Preferably, the bite block 100 is sized such that the patient's molars are in contact with the bite block 100.

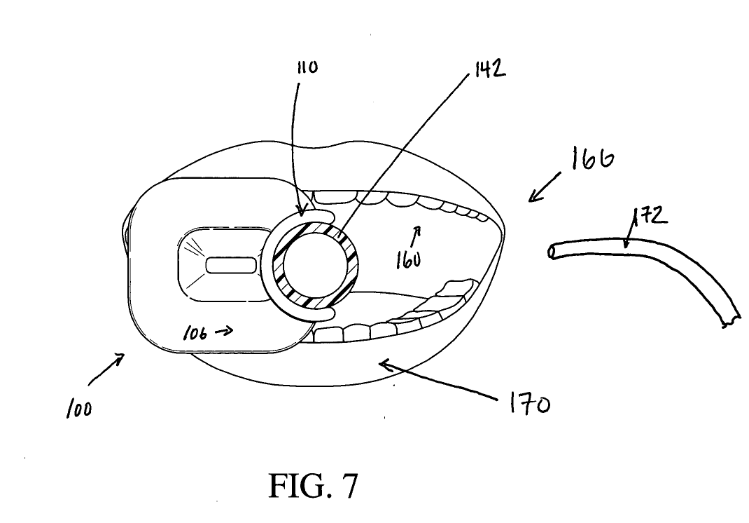

FIG. 7 illustrates a front view of an LMA tube 142 which has been inserted into a patient's oral cavity 160, wherein the bite-block 100 has been attached to the LMA tube 142. FIG. 7 demonstrates that, in one embodiment, the flange 106 rests in front of a significant fraction of the total area of the patient's lips 170. As a result, the bite-block 100 is substantially constrained from entering the oral cavity 160, enhancing the safety of use of the device 100. Further, when the LMA tube 142 is placed within the coupling mechanism 110 of the bite-block 100, the tube 142 is approximately centered within the patient's mouth 166. Because the tube 142 emerges from the patient's throat at approximately the center of the mouth 166 when in use, the tube 142 does not have to be moved significantly in order to couple the tube 142 to the bite-block 100, substantially reducing any discomfort experienced by the patient in installing the bite-block. In addition, once in place, the LMA tube 142 may also be secured to the patient using adhesive tape (not shown) to further secure the tube 142.

Also illustrated in FIG. 7 is the size of the bite-block device 100 with respect to the patient's mouth 166 and oral cavity 160 in one embodiment of the invention. The bite-block 100 and LMA tube 142 occlude roughly half of the opening of the mouth 166, preserving line-of-sight into the oral cavity 160. During oral procedures, it is critical to remove regurgitation and fluids within the mouth during oral procedures. This task is generally accomplished through use of suction devices, often long, narrow tubes connected to a vacuum apparatus. These suction devices are classified as soft or hard, depending on the relative flexibility of the suction tube, with relatively flexible tubes designated “soft” and relatively rigid tubes designated “hard.” FIG. 7 illustrates that the unobstructed area of access to within the oral cavity 160 allows insertion of a suction tube 172 connected to a hard or soft suction device (not shown) into the oral cavity 160 in order to perform important fluid removal. Line of sight into the cavity also allows visual manipulation of suction devices while the bite-block 100 and the LMA tube 142 are inserted in the oral cavity 160, allowing the physician greater control over suction devices inserted into the mouth and enhancing the safety of oral procedures.

Although the foregoing description has shown, described, and pointed out the fundamental novel features of the present teachings, it will be understood that various omissions, substitutions, and changes in the form of the detail of the apparatus as illustrated, as well as the uses thereof, may be made by those skilled in the art, without departing from the scope of the present teachings. Consequently, the scope of the present teachings should not be limited to the foregoing discussion, but should be defined by the appended claims.

What is claimed is:

1. A bite block apparatus for use with LMA air tubes when inserted into a patient, the apparatus comprising:
a bite member having a first and a second end wherein the bite member is dimensioned so as to engage a set of teeth of the patient positioned on a first side of the patient's mouth so that the patient is inhibited from closing their mouth to a position wherein the patient's front teeth are less than a first distance apart; and
a tube retention member coupled to the first end of the bite member such that the tube retention member is positioned outside of the patient's mouth when the bite member is engages the set of the teeth on the first side of the patient mouth, wherein the tube retention member is sized so as to receive the LMA air tube such that the LMA air tube extends into the patient mouth in between the patient's front teeth.

2. The apparatus of claim 1, wherein the bite member is dimensioned so as to be interposed between the patient's molars which the tube retention member being positioned at approximately the center of the patient's mouth.

3. The apparatus of claim 2, wherein the bite member defines two surfaces that engage the patient's molars wherein the surfaces are approximately ¾ inches wide by 1¼ inches long and are spaced apart by approximately 9/16 of an inch.

4. The apparatus of claim 3, wherein the bite member is tapered from the first end to the second end so as to engage the patient's molars in a spaced apart configuration.

5. The apparatus of claim 1, further comprising a flange positioned adjacent the first end of the bite member wherein the flange engages with the lips of the patient so as to retain the tube retention member outside of the patient's mouth.

6. The apparatus of claim 5, wherein the tube retention member is integrally coupled to the flange so as to extend laterally outwards therefrom.

7. The apparatus of claim 6, wherein the flange and bite member define a through going hole that extends the length of the apparatus to allow air to flow therethrough.

8. A device for securing an LMA air tube in the mouth of a patient, the device comprising:
a bite member having a first and a second end and defining a first and second bite surfaces dimensioned so as to engage with a set of side teeth of he patient and retain the side teeth spaced from each other;
a flange member coupled to the first end of the bite member wherein the bite member and flange member are dimensioned such that the flange member is positioned outside of the patient's mouth when the bite member is engaging with the set of side teeth of the patient;
a tube securing member coupled to the flange member wherein the tube securing member is sized so as to receive the LMA tube and retain the LMA tube in substantially a first position in the patient's mouth and wherein the patient's front teeth are inhibited from damaging the LMA tube as a result of the engagement of the patient's teeth with the first and second bite surfaces.

9. The apparatus of claim 8, wherein the bite member is dimensioned so as to be interposed between the patient's molars which the tube retention member being positioned at approximately the center of the patient's mouth.

10. The apparatus of claim 9, wherein the bite surfaces that engage the patient's molars wherein the surfaces are approximately ¾ inches wide by 1¼ inches long and are spaced apart by approximately 9/16 of an inch.

11. The apparatus of claim 10, wherein the bite member is tapered from the first end to the second end so as to engage the patient's molars in a spaced apart configuration.

12. The apparatus of claim 8, wherein the flange positioned adjacent the first end of the bite member so that the flange engages with the lips of the patient so as to retain the tube securing member outside of the patient's mouth.

13. The apparatus of claim 13, wherein the tube securing member is integrally coupled to the flange so as to extend laterally outwards therefrom.

14. The apparatus of claim 6, wherein the flange and bite member define a through going hole that extends the length of the apparatus to allow air to flow therethrough.

15. A method of preventing damage to an air tube of an LMA device, the method comprising:
inserting the LMA device into the patient wherein the LMA device includes an air tube that supplies air to the patient from an external source via the LMA device;
positioning a bite block member between a plurality of the patient's molars on a first side of the patient's mouth so that the patient's front teeth are maintained a first distance apart;
coupling the LMA air tube to a retainer formed on the bite block member such that the LMA air tube is positioned at substantially the center of the patient's mouth with the air tube being interposed between the patient's front teeth that are maintained the first distance apart.

16. The method of claim 15, wherein coupling the LMA air tube the retainer comprises positioning the LMA air tube into a hemispherical opening that is attached to the bite block at a location outside of the patient's mouth.
